(19) United States
(12) Patent Application Publication
(10) Pub. No.: US 2011/0021996 A1
(43) Pub. Date: Jan. 27, 2011

(54) STRUCTURE OF MICRO-NEEDLE WITH SIDE CHANNEL AND MANUFACTURING METHOD THEREOF

(75) Inventors: Seung Seob Lee, Daejeon (KR); Boo Joon Sul, Daejeon (KR); Man Hee Han, Daejeon (KR)

Correspondence Address: Edwards Angell Palmer & Dodge LLP, P.O. Box 55874, Boston, MA, 02205, US

(73) Assignee: MITI SYSTEMS INC., Daejeon (KR)

(21) Appl. No.: 12/895,378

(22) Filed: Sep. 30, 2010

Related U.S. Application Data
(63) Continuation of application No. PCT/KR2009/001146, filed on Mar. 9, 2009.

(30) Foreign Application Priority Data
Dec. 18, 2008 (KR) 10-2008-129332
Aug. 4, 2009 (KR) 10-2009-0071572
May 13, 2010 (KR) PCT/KR2010/003022

(51) Int. Cl. A61M 5/00; A61M 5/32

(52) U.S. Cl. 604/173; 604/272; 427/2.28

(57) ABSTRACT

The present invention relates to a microneedle for delivering active agent or agents. The microneedle comprises a body a body having a base at one end thereof and a tip portion at the other end thereof; a pair of projections connected to a side of the body and having a base at one end thereof and a tip portion at the other end thereof; and a vertical groove defined by the body and the pair of projections. The distance between outer edges of the pair of projections is formed to become smaller towards the tip portion than the distance between outer edges of the body, and the upper end of each of the pair of projections is shorter than that of the body such that a free space is formed between the outer edges of the body and the pair of projections.

### CROSS-REFERENCE TO RELATED APPLICATION

This is a continuation of International Application No. PCT/KR2009/001146, with an international filing date of Mar. 9, 2009, which claims the benefit of Korean Application No. 10-2008-129332 filed Dec. 18, 2008, the entire contents of which are incorporated herein by reference.

### TECHNICAL FIELD

The present invention relates to a microneedle having a fluid passageway formed along a side thereof, a method for manufacturing the same, and an in-plane and an out-of-plane microneedle array comprising the same.

### BACKGROUND ART

Methods of painless, simple, and convenient delivery of active agents or ingredients (e.g., cosmetic or medical active agent) into human body through the skin texture have been proposed. One of the obstacles to application of these transdermal delivery methods is that since the stratum corneum, an outermost layer of the epidermis of the skin, is 10-60 μm in depth inhibits the outflow of internal body substances and the penetration of external substances into the human body, transdermal absorption of active ingredients is low. Particularly, if the active ingredients are hydrophilic or have a large molecular weight, the transdermal absorption thereof further decreases.

Conventionally, needles which have a diameter measured in millimeter units (mm) and a length measured in centimeter units (cm) have been used for these transdermal delivery. Such conventional needles, however, stimulate a plurality of pain spots widely distributed in the skin, which gives a considerable pain to a subject in use.

In order to address and solve the above problem, microneedles have been developed which have a diameter of several tens to several hundreds of micrometers (μm) and a length of several hundreds to several thousands of micrometers (μm). Since these microneedles are relatively small in diameter and length as compared to the conventional needles, the number of pain spots stimulated is reduced, thereby resulting in significant alleviation of a pain given to the subject.

Since the use of one microneedle decreases drug delivery efficiency or greatly reduces the amount of extractable internal body substances, the microneedles are configured in an array in which a plurality of microneedles is arranged according to a predetermined arrangement pattern as shown in FIGS. 1 and 2. The microneedle array is divided into an in-plane microneedle array and an out-of-plane microneedle array depending on whether the arrangement of each microneedle is two-dimensional or three-dimensional.

In the meantime, there have been developed microneedle structures which are diversely modified so as to enhance percutaneous delivery efficiency of an active agent or agents using such a microneedle array.

U.S. Pat. Nos. 3,964,482 and 6,256,533 disclose a hollow microneedle having a fluid passageway or channel formed therein so as to allow an active agent or agents to be transferred into human body therethrough. However, since such a hollow microneedle has a diameter of several tens to several hundreds of micrometers and requires that it should have a fluid channel of a smaller diameter formed therein, it can be fabricated only by a highly delicate work. Furthermore, this type of microneedle has a shortcoming that since a core used for forming the channel is relatively long as compared to the diameter of the microneedle, it can be deformed due to a force generated by the flow of a fluid or a pressure applied to a mold, which may occur during the manufacturing process.

U.S. Pat. No. 6,881,203 discloses a microneedle array including a plurality of microneedles each having a fluid channel formed in the outer surface of each microneedle and being in fluid communication with an optional conduit structure formed on the substrate surface along each row of microneedles. The '203 patent teaches a method of fabricating a mold (out-of-plane microneedle array) in such a fashion as to engrave a substrate in the shape of three-dimensional microneedles to form cavities in the shape of the desired microneedles by using various processes such as laser ablation, photolithography, chemical etching, ion beam etching, etc. However, according to the above method, since the engravings such as the cavities must be formed as many as the number of microneedles to be installed on the microneedle array as well as a three-dimensional engraving must be performed, it is not easy to obtain various structures of more sophisticated microneedles.

Korean Patent No. 682,534 to the present inventors discloses a method for manufacturing a sophisticated and low-priced out-of-plane microneedle array which comprises performing photolithography and electroplating processes on a planar base to fabricate an in-plane microneedle array, and then vertically arranging the in-plane microneedle array on a planar substrate to fabricate the out-of-plane microneedle array (see FIGS. 3 and 4). Furthermore, the '534 patent teaches a method for forming a vertical groove along a side of each microneedle in a longitudinal direction of the microneedle in order to facilitate the injection of a drug into human body or the extraction of internal body substances. Specifically, as shown in FIGS. 5 and 6, the '534 patent suggests a method of using a base 100 having a plurality of trenches 103 formed thereon by a variety of etching methods, a method of forming trenches 103 on the base 100 before the electroplating is performed, and a method of directly forming the vertical groove along the side of each microneedle using laser machining or electrical discharge machining (EDM).

However, since the microneedle formed with the vertical groove fabricated by the conventional methods has a low precision, it requires an inconvenient and difficult process in which the outer appearance of the microneedle is surface-finished sophisticatedly after a primary fabrication of the microneedle.

In the meantime, intracutaneous absorption rate of the active ingredients by the microneedle is determined depending on how fast the active ingredients from the outside of human body are transferred to a skin depth where a diffusion rate thereof is high, and how fast the active ingredients are diffused into the human body. The microneedle formed with the vertical groove is provided to improve the active ingredient-transferring effect in the former. One method for improving the active ingredient-diffusing effect in the latter is to increase the area where the active ingredients are in contact with the skin. In the microneedle formed with the vertical groove, an increase in the width of the groove results in an increase in the area where the active ingredients are in contact with the skin. However, since the skin texture is high in elasticity, the skin serves to obstruct or clog the vertical groove, which results in a decrease in the speed at which the active ingredients are transferred to a skin depth where a diffusion rate thereof is high.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

In one aspect, the present invention provides a microneedle for delivering active agent or agents. The microneedle comprises a body, a pair of projections connected to a side of the body, and a vertical groove defined by the body and the pair of projections. The body has a base at one end thereof and a tip portion at the other end thereof. The pair of projections has a base at one end thereof and a tip portion at the other end thereof. In the microneedle, the distance between outer edges of the pair of projections is formed to become smaller towards the tip portion than the distance between outer edges of the body, and the upper end of each of the pair of projections is shorter than that of the body such that a free space is formed between the outer edges of the body and the pair of projections.

In certain embodiments, the body and the pair of projections may be formed integrally. In another certain embodiments, the body, the base thereof, and the tip portion thereof may suitably be formed integrally. In addition, the pair of projections, the base thereof, and the tip portion thereof may suitably be formed integrally.

In another aspect, the present invention provides a method for manufacturing the microneedle. The method comprises: (a) forming on a light-transmitting substrate a light-shielding pattern having the shape of a planar cross-section of the microneedle; (b) coating the light-transmitting substrate with a photoresist, masking the photoresist in a light-shielding pattern corresponding to the vertical groove, and exposing the masked photoresist to light to thereby define projections for the vertical groove; (c) coating the light-transmitting substrate with a photoresist, exposing and developing the photoresist from the bottom surface of the light-transmitting substrate to thereby form a mold for the microneedle formed with the vertical groove; (d) depositing an electroplating seed layer on the top surface of the mold to form a plating layer; and (e) removing the plating layer by performing a plating process on the electroplating seed layer.

In certain embodiments, the light-shielding pattern corresponding to the vertical groove in the step (b) may be configured in such a fashion that a slit corresponding to the vertical groove is formed in a tip of the microneedle, and the width and length thereof are smaller by a predetermined dimension than those of the light-shielding pattern having the planar cross-section shape of the microneedle in the step (a). In these embodiments, preferably, the light-shielding pattern having the planar cross-section shape of the microneedle and the light-shielding pattern corresponding to the vertical groove may become narrower as they go toward the top.

In another certain embodiments, in the step (a), the light-shielding pattern may have the shape of a planar cross-section of a tip of the microneedle. In this case, the step (c) may comprise exposing the photoresist to light from the bottom surface of the light-transmitting substrate, placing a light-shielding pattern having the shape of a planar cross-section of a base of the microneedle on the top surface of the light-transmitting substrate, and exposing and developing the photoresist from the top surface of the light-transmitting substrate.

In further embodiments, the light-shielding pattern in the step (a) may have the shape of the planar cross-section of one or more microneedles which are arranged in parallel with each other. In these embodiments, the exposing of the photoresist in the step (c) may be performed in an inclined manner.

In still further embodiments, the method may further comprise the steps of: (f) fabricating a negative mold using the removed plating layer as an original form; and (e) obtaining a molded product made of a polymer material using the negative mold.

In still another aspect, the present invention provides an in-plane and an out-of-plane microneedle sheet comprising a plurality of the above-described microneedles.

In certain embodiments, the sheet may comprise a through-recess formed therein so as to store or transport active agent or agents. The through-recess is adapted to fluidically communicate with the vertical groove of the microneedle.

Terminology herein is merely used to describe specific embodiments of the invention, but is not intended to limit the invention. The singular forms “a”, “an” and “the” include plural referents unless the context clearly dictates otherwise.

Hereinafter, in the present invention, a distal end of a microneedle is defined as a “tip”. A bottom portion of the microneedle is defined as a “base”. Sidewalls defining the vertical groove are defined as “projections”. A portion which extends beneath the tip in such a fashion as to define a bottom of the vertical groove and abut against the rear surface of the projections is defined as a “body”. Also, the sides of the projections and the body are defined as an “outer edges”. In addition, the terms “upper portion” and “lower portion” of each microneedle as defined herein refer to a tip portion and a base portion which are in a relative positional relationship, respectively. The terms “upper surface” and “lower surface” of each microneedle as defined herein refer to a top surface of the projections defining the vertical groove and a bottom surface of the body opposite to the projections, respectively.

The above and other features and advantages of the present invention will be apparent from or are set forth in more detail in the accompanying drawings, which are incorporated in and form a part of this specification, and the following Detailed Description, which together serve to explain by way of example the principles of the present invention.

### BRIEF DESCRIPTION OF THE DRAWINGS

FIG. 1 is a perspective view illustrating an example of an in-plane microneedle array;

FIG. 2 is a perspective view illustrating an example of an out-of-plane microneedle array;

FIG. 3 is a flowchart illustrating a process of manufacturing an in-plane microneedle array according to the prior art;

FIG. 4 is a flowchart illustrating a process of manufacturing an out-of-plane microneedle array according to the prior art;

FIGS. 5 and 6 are perspective views illustrating a base having trenches formed thereon according to the prior art;

FIGS. 7 and 8 are perspective views illustrating an example of a microneedle according to the present invention and an example of a microneedle according to the prior art, respectively;

FIGS. 9 and 10 each illustrate an example of a process for fabricating a mold for a microneedle having a vertical groove according to the present invention;

FIG. 11 is a transverse cross-sectional view illustrating another example of a process for manufacturing a microneedle formed with a vertical groove according to the present invention;

FIGS. 12 and 13 are conceptual views illustrating the case where the pattern corresponding to a vertical groove protrudes outwardly from the pattern of a tip of the microneedle according to the present invention and a photograph of the microneedle manufactured by a method of manufacturing the same according to the present invention, respectively;

FIGS. 14 and 15 are conceptual views illustrating the case where the pattern corresponding to a vertical groove is positioned within the pattern of a tip of the microneedle according to the present invention and a photograph of the microneedle manufactured by a method of manufacturing the same according to the present invention, respectively; and

FIG. 16 is a photograph illustrating an example of the manufacturing of an out-of-plane microneedle sheet to which the microneedle of the present invention is to be applied.

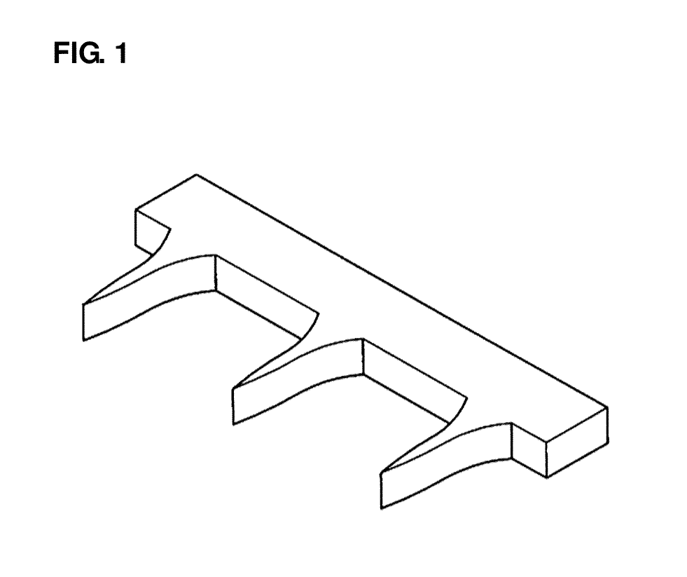

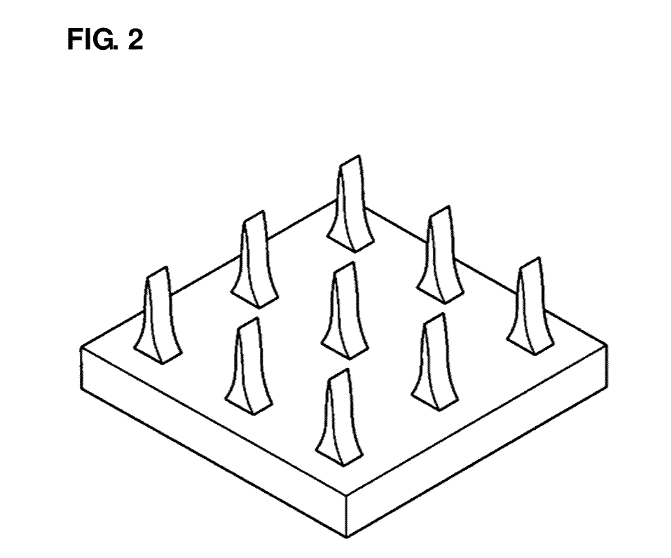

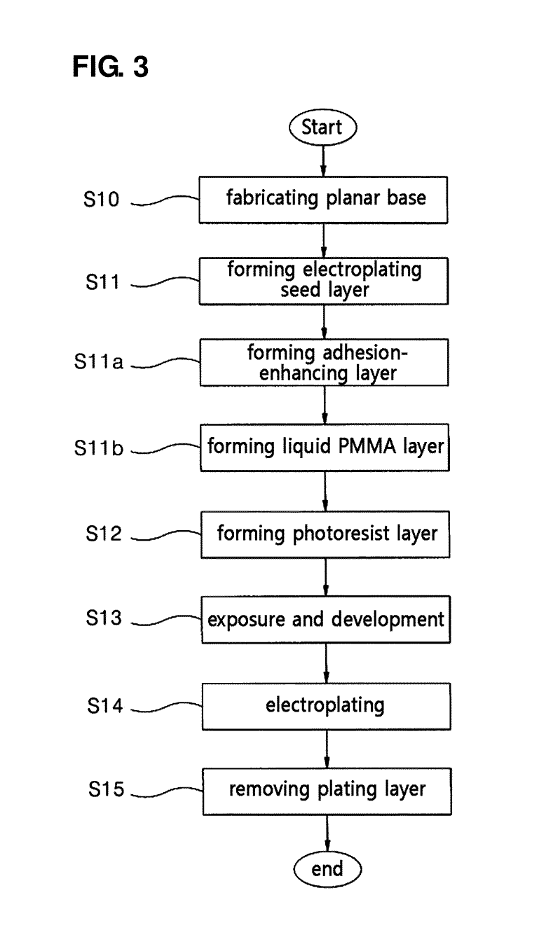

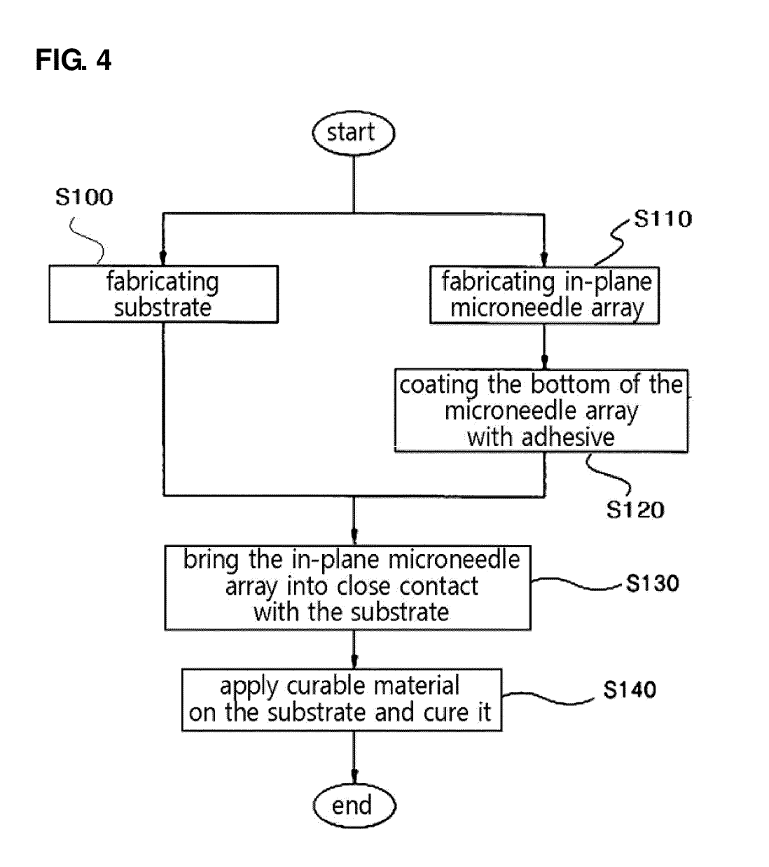

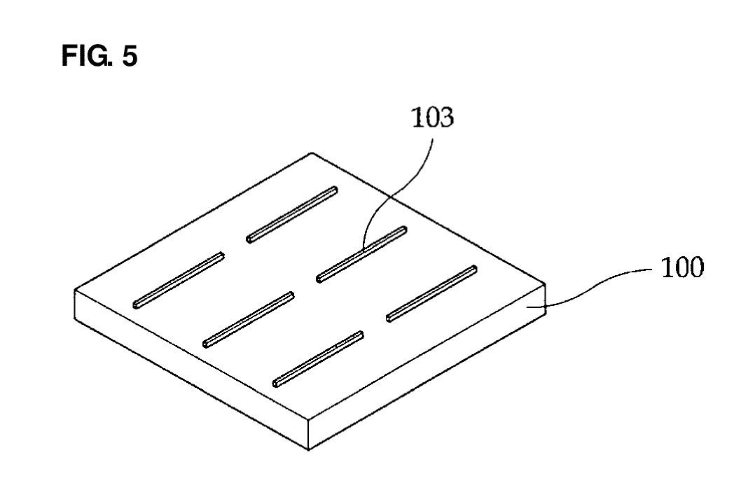

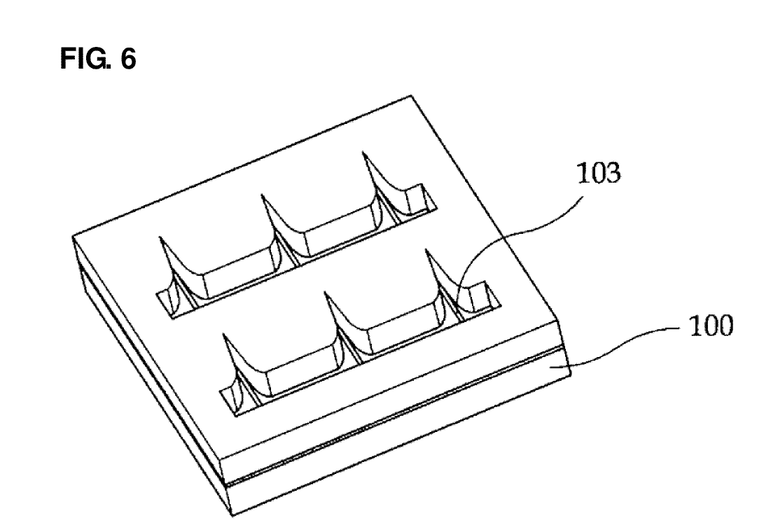

### DETAILED DESCRIPTION OF THE DISCLOSURE

The present invention will be hereinafter described in detail in connection with the preferred embodiments with reference to the accompanying drawings. However, these embodiments of the present invention are merely illustrative of easy explanation on the contents and scope of the technical spirit of the present invention, but the technical scope of the present invention is not limited or modified thereby. Also, it will be understood by those skilled in the art that various modifications and variations can be made to the present invention without departing from the spirit and scope of the appended claims based on the illustrative embodiments.

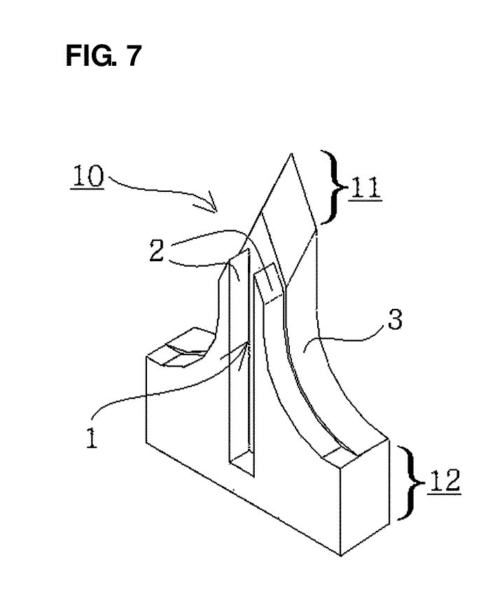

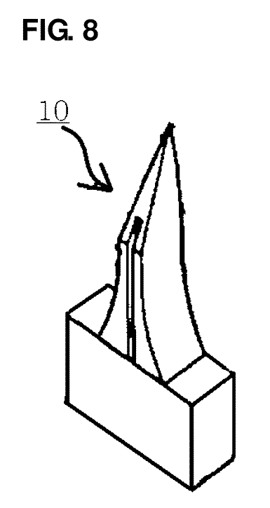

FIGS. 7 and 8 are perspective views illustrating an example of a microneedle according to the present invention and an example of a microneedle according to the prior art, respectively.

The microneedle according to the prior art as shown in FIG. 8 is constructed such that a vertical groove 1 is simply formed at a side of an integral body 3. The integral microneedle enables active ingredients to be rapidly transferred to a skin depth. However, if the width of the vertical groove 1 increases so as to increase the area where active ingredients are in close contact with a skin, the skin may be partially pushed forcedly into the vertical groove 1, thereby causing the vertical groove 1 to be obstructed or clogged.

On the other hand, as shown in FIG. 7, the microneedle according to the present invention comprises a body having a base at one end thereof and a tip portion at the other end thereof, a pair of projections connected to a side of the body and having a base at one end thereof and a tip portion at the other end thereof, and a vertical groove defined by the body and the pair of projections. The distance between outer edges of the pair of projections is formed to become smaller than the distance between outer edges of the body and the upper end of each of the pair of projections is shorter than that of the body such that a free space is formed between the outer edges of the body and the pair of projections.

Preferably, the pair of projections 2 and the body 3 may be integrally formed. Also preferably, the pair of projections 2, the base thereof, the tip portion thereof may be integrally formed Also preferably, the body 3, the base thereof, the tip portion thereof may be integrally formed.

When the microneedle of the present invention is applied to the skin, the active ingredients transferred along the vertical groove 1 can reach the free space formed between the outer edges of the body 3 and the projections 2 adjacent to the tip 11. In this case, since the free space increases the area in close contact with the skin texture, it can enhance the efficiency of the diffusion of the active ingredients diffuse into the human body.

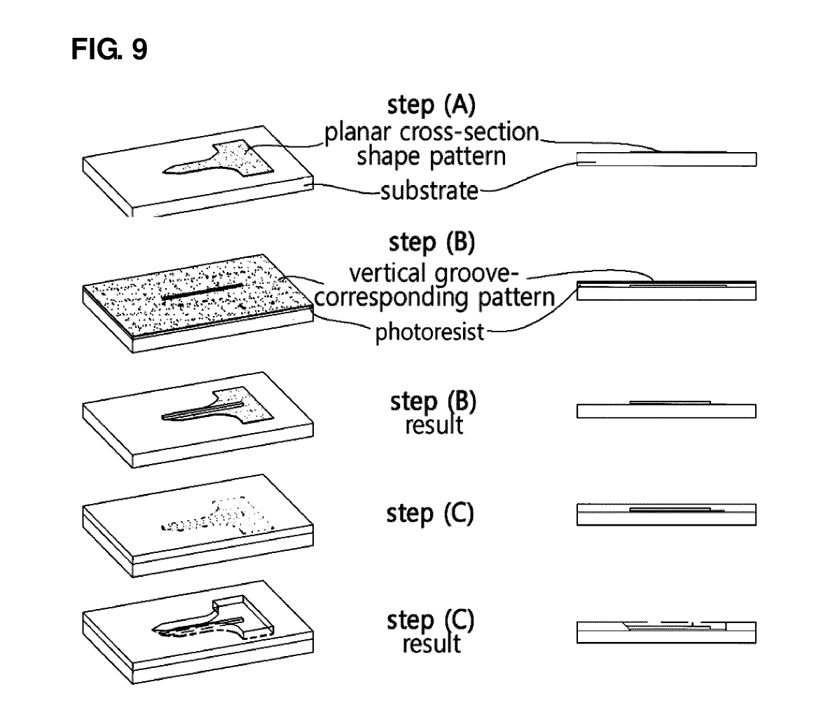

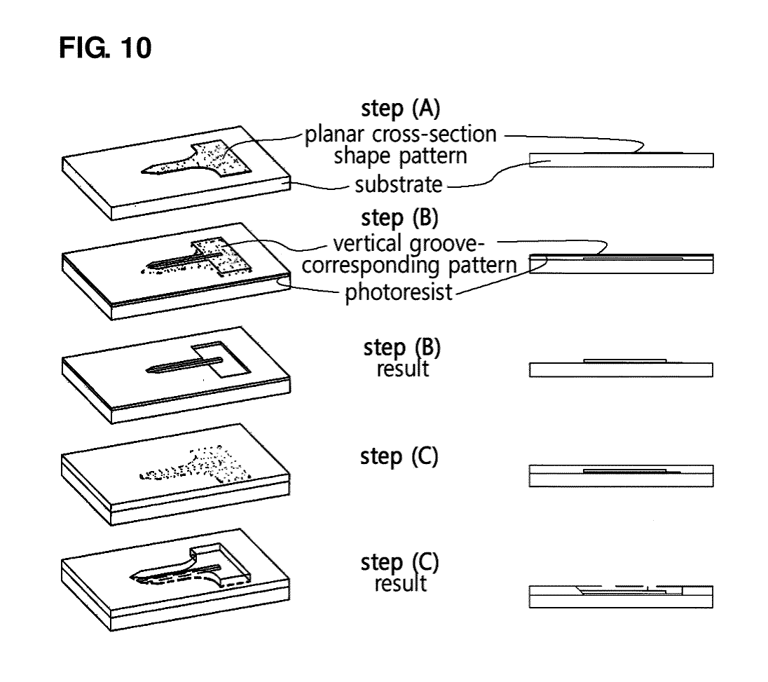

FIGS. 9 and 10 each illustrate an example of a process for fabricating a mold for a microneedle having a vertical groove according to the present invention. The processes shown in FIGS. 9 and 10 have the same steps except a difference in the structure of a light-shielding pattern, which corresponds to the vertical groove, in the step (B). In FIGS. 9 and 10, the figures on the left side are perspective views of the mold for the microneedle and the figures on the right side are longitudinal cross-sectional views of the mold.

In more detail, in the step (A), a light-shielding pattern having the shape of a planar cross-section of the microneedle is formed on a light-transmitting substrate.

Subsequently, in the step (B), a photoresist is coated to a thickness corresponding to a depth of the vertical groove 1 on the light-transmitting substrate, and the photoresist is exposed to light using a mask having a light-shielding pattern corresponding to the vertical groove 1 to thereby define projections 2 for the vertical groove 1.

In the step (C), a photoresist is coated to a thickness the same as that of the microneedle on the light-transmitting substrate formed with the projections for the vertical groove 1, and then the photoresist is exposed to light and developed from the bottom surface of the light-transmitting substrate. Through this process, a mold for the microneedle formed with the vertical groove can be obtained.

Thereafter, although not shown, an electroplating seed layer is deposited on the top surface of the obtained mold to form a plating layer (step (D)), and then the plating layer is removed by performing a plating process on the electroplating seed layer (step (E)) to thereby obtain the microneedle formed with the vertical groove 1. Thereafter, a finishing process may be additionally performed in which the removed plating layer is subjected to grinding, lapping and polishing, if necessary.

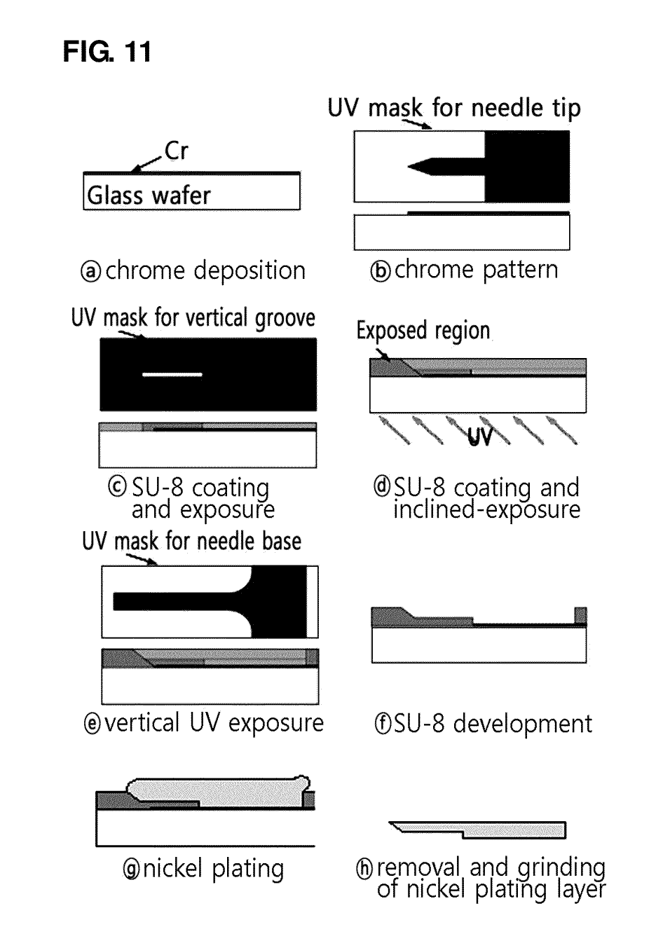

FIG. 11 is a transverse cross-sectional view illustrating another example of a process for manufacturing the microneedle formed with a vertical groove according to the present invention.

In the microneedle manufacturing process of FIG. 11, which will be described in detail below, the light-shielding pattern is divided into a pattern corresponding to the tip 1 of the microneedle and a pattern corresponding to the base 12 of the microneedle. In more detail, in the step (A), after the shape of the microneedle tip 11 is established by using the light-shielding pattern having the shape of a planar cross-section of the tip 11 of the microneedle, the photoresist is exposed to light from the bottom surface of the light-transmitting substrate and a light-shielding pattern having the shape of a planar cross-section of the base 12 of the microneedle is placed on the top surface in the step (C). Thereafter, the photoresist is exposed to light and developed from the top surface of the light-transmitting substrate to thereby obtain the mold for microneedle formed with the vertical groove 1. The subsequent steps are the same.

### Embodiments

The present invention will be illustrated with reference to the following Embodiments. In the following Embodiments, an ultraviolet ray was used as a light source for use in the exposure of the photoresist, a glass substrate was used as the light-transmitting substrate, a negative UV photoresist was used as the photoresist, a titanium layer was used as the electroplating seed layer, and a nickel plating process was used as the plating process. But it is natural that the same result can be obtained through the selection of diverse methods using a variety of materials. Thus, the description of various modifications will be omitted hereinafter. In addition, since an in-plane microneedle array and an out-of-plane microneedle array can be easily manufactured based on the microneedle manufactured according to the present invention with reference to the prior art, detailed description thereof will be omitted. Materials and methods necessary to implement the present invention refer to those described in Korean Patent No. 10-0682534, which is incorporated by reference herein, to the present inventors. The following Embodiments are provided solely for illustration purpose only without limiting the scope of the present invention.

### Embodiment 1

### Microneedle Manufacturing Process Adopting the Light-Shielding Pattern Divided into a Pattern Corresponding to the Tip of the Microneedle and a Pattern Corresponding to the Base of the Microneedle (see FIG. 11)

Referring to FIG. 11, UV-shielding chrome (Cr) was deposited on a glass substrate through which a ultraviolet ray can transmit, and the chrome-deposited layer was subjected to a photolithography process which employs a UV mask for a microneedle tip pattern defining the shape of the tip of the microneedle to thereby form the shape of a pointed tip of the microneedle on the chrome-deposited layer. (steps \textcircled{a} and \textcircled{b}).

Subsequently, SU-8 as a negative photoresist was coated to a desired depth of the vertical groove on the substrate, and then was exposed and heat-treated using a UV mask for a vertical groove pattern defining the shape of the vertical groove of the microneedle. Then, the photoresist region exposed to the UV light was cured and was not removed upon the development in a post-process to thereby form the projections for the vertical groove (step \textcircled{c}).

Thereafter, SU-8 was coated to a thickness larger than a desired thickness of the microneedle on the substrate, and then was inclinedly exposed to the UV light from the bottom of the glass substrate. When the UV inclined-exposure process was performed, the pattern shape of the chrome-deposited layer served as a mask so that a three-dimensional microneedle tip shape can be defined (step \textcircled{d}).

Next, a UV mask for a microneedle base pattern defining the shape of the base of the microneedle was placed on the substrate coated with the SU-8 photoresist, which was in turn exposed and heat-treated, followed by development. Then, the SU-8 photoresist region not exposed to the UV light was removed to thereby obtain a negative mold having the shape of a microneedle (steps \textcircled{e} and \textcircled{f}).

An electroplating seed layer was deposited on the mold and then was subjected to a nickel plating process (step \textcircled{g}). Then, a nickel plating layer was removed from the substrate and an excessively thick plated portion was eliminated using a grinding process to thereby fabricate a microneedle having a fluid passageway formed along a side thereof (step \textcircled{h}).

### Embodiment 2

### Microneedle Manufacturing Process Adopting a Pattern Corresponding to the Entire Shape of the Microneedle (see FIG. 11)

In Embodiment 2, the microneedle was manufactured in the same process as that used in Embodiment 1 except that a UV mask for the patterns defining the entire shape including the tip and the base of the microneedle is used in the steps \textcircled{a} and \textcircled{b} the steps \textcircled{e} and \textcircled{f} are omitted. In the step \textcircled{d}, preferably, the tip side of the microneedle may be subjected to the inclined-exposure and the base side of the micneedle may be subjected to the vertical exposure according to circumstances.

### Embodiment 3

### Microneedle Manufacturing Process Adopting a Different Positional Relationship between the Tip Pattern and the Vertical Groove Pattern of the Microneedle

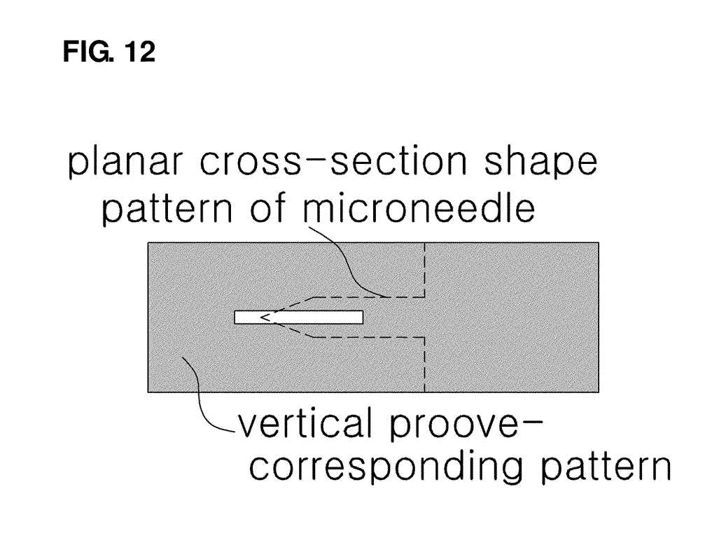

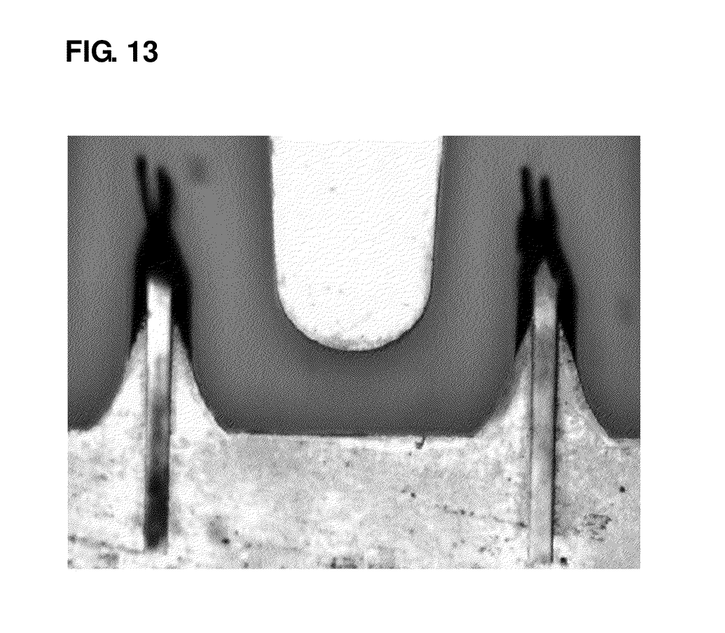

The microneedle was fabricated in the same manner as that used in the Embodiment 1 or 2. In Embodiment 3, the vertical groove pattern is formed to extend beyond the tip pattern (see FIGS. 9 and 12). Since an unnecessary nickel film is formed at the tip of the microneedle, a work for finishing this was required.

As shown in FIG. 12, the nickel film formation occurs due to the fact that a mask pattern (indicated by doted line) defining the shape of the tip of the microneedle and a mask pattern (indicated by solid line) defining the shape of the vertical groove of the microneedle cross each other. In other words, when a photoresist is coated to a thickness corresponding to a depth of the vertical groove on the light-transmitting substrate and is exposed to light, followed by the heat treatment, the exposed portion of the photoresist layer is cured, which results in a difference in refractive index between the cured portion and the non-cured portion. In the subsequent step \textcircled{d}, when the UV light is slantly incident to the SU-8 photoresist from the bottom of the glass substrate to define the shape of the microneedle tip, the path of light is changed at the interface between the cured portion and the non-cured portion, so that since an extended portion of the interface is not exposed to the UV light, the photoresist of this light-unexposed portion is not cured accordingly and is dissolved in a developing solution. Then, nickel is plated thereon to thereby form a nickel film.

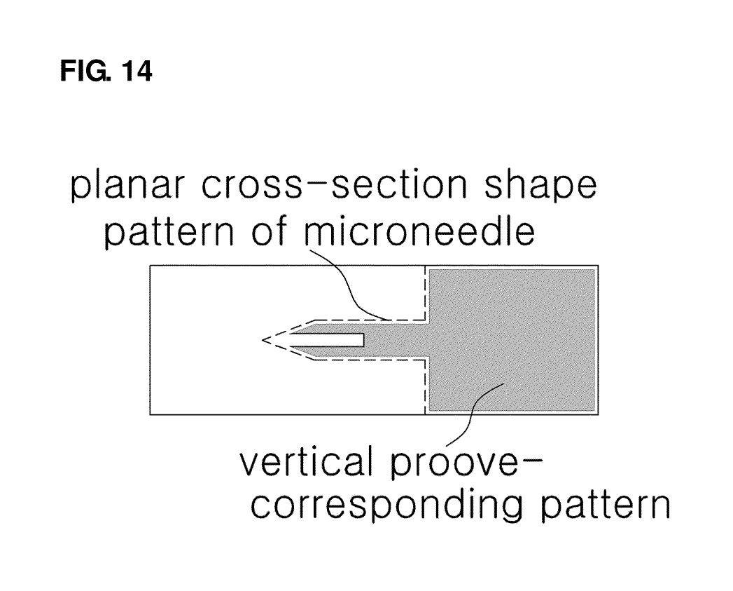

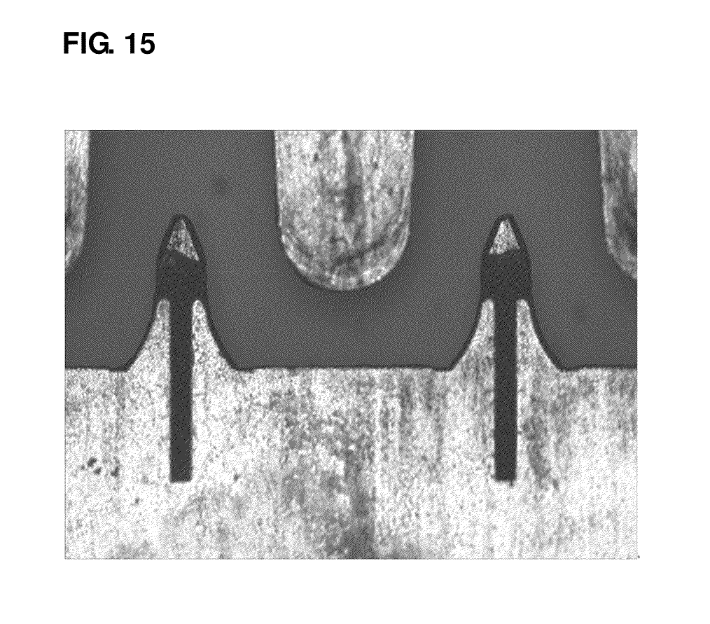

In order to prevent such an unnecessary nickel film, the mask pattern (indicated by solid line) for the vertical groove of the microneedle was positioned within the mask pattern (indicated by doted line) for the microneedle tip (see FIGS. 10 and 14) to thereby fabricate the microneedle in the same method as in Embodiment 1 or 2 (see FIG. 15).

As a result, an unnecessary nickel film was no longer formed, so that the microneedle formed with the vertical groove for a fluid passageway could be fabricated, which eliminates the necessity of a subsequent finishing work

### Embodiment 4

### Manufacture of Out-of-plane Microneedle Sheet

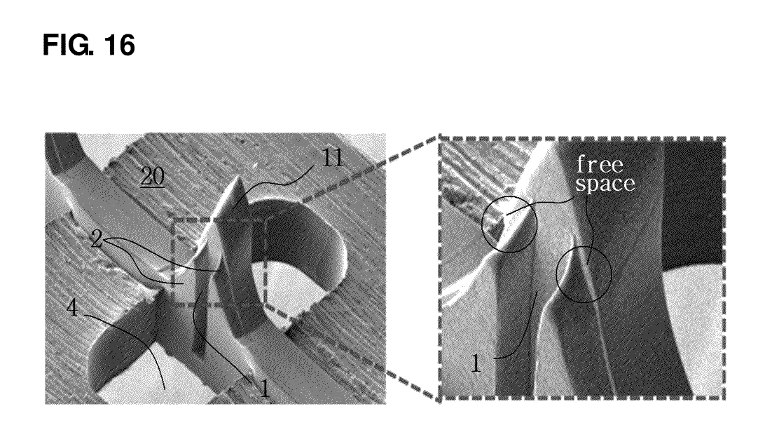

Using the microneedle fabricated in the above-mentioned Embodiments, an out-of-plane microneedle sheet 20 was manufactured. As shown in FIG. 16, the out-of-plane microneedle sheet 20 includes a through-recess 4 formed therein so as to store or transport a active agents, the through-recess being adapted to fluidically communicate with the vertical groove 1 of the microneedle. Since the manufacturing method of the out-of-plane microneedle sheet 20 is known in the art, the detailed description thereof will be omitted.

As described above, according to the present invention, a microneedle formed with a micro-vertical groove, which can easily transcutaneously deliver a drug without a pain, can be manufactured only through exposure, development and plating processes. In addition, since the microneedle is fabricated in a state where the tip thereof has been finished, a process of finishing the manufactured product can also be omitted, thereby simplifying the production process and increasing the quality of the final product.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A microneedle for delivering active agent or agents, the microneedle comprising:
a body having a base at one end thereof and a tip portion at the other end thereof;
a pair of projections connected to a side of the body and having a base at one end thereof and a tip portion at the other end thereof; and
a vertical groove defined by the body and the pair of projections;
wherein the distance between outer edges of the pair of projections is formed to become smaller towards the tip portion than the distance between outer edges of the body, and the upper end of each of the pair of projections is shorter than that of the body such that a free space is formed between the outer edges of the body and the pair of projections.

2. The microneedle according to claim 1, wherein the body and the pair of projections are formed integrally.

3. The microneedle according to claim 1, wherein the body, the base thereof, and the tip portion thereof are formed integrally.

4. The microneedle according to claim 1, wherein the pair of projections, the base thereof, and the tip portion thereof are formed integrally.

5. A method for manufacturing the microneedle according to claim 1, comprising the steps of:
(a) forming on a light-transmitting substrate a light-shielding pattern having the shape of a planar cross-section of the microneedle;
(b) coating the light-transmitting substrate with a photoresist, masking the photoresist in a light-shielding pattern corresponding to the vertical groove, and exposing the masked photoresist to light to thereby define projections for the vertical groove;
(c) coating the light-transmitting substrate with a photoresist, exposing and developing the photoresist from the bottom surface of the light-transmitting substrate to thereby form a mold for the microneedle formed with the vertical groove;
(d) depositing an electroplating seed layer on the top surface of the mold to form a plating layer; and
(e) removing the plating layer by performing a plating process on the electroplating seed layer.

6. The method according to claim 5, wherein the light-shielding pattern corresponding to the vertical groove in the step (b) is configured in such a fashion that a slit corresponding to the vertical groove is formed in a tip of the microneedle, and the width and length thereof are smaller by a predetermined dimension than those of the light-shielding pattern having the planar cross-section shape of the microneedle in the step (a).

7. The method according to claim 6, wherein the light-shielding pattern having the planar cross-section shape of the microneedle and the light-shielding pattern corresponding to the vertical groove become narrower as they go toward the top.

8. The method according to claim 5, wherein in the step (a), the light-shielding pattern has the shape of a planar cross-section of a tip of the microneedle, and the step (c) comprises exposing the photoresist to light from the bottom surface of the light-transmitting substrate, placing a light-shielding pattern having the shape of a planar cross-section of a base of the microneedle on the top surface of the light-transmitting substrate, and exposing and developing the photoresist from the top surface of the light-transmitting substrate.

9. The method according to claim 5, wherein the light-shielding pattern in the step (a) has the shape of the planar cross-section of one or more microneedles which are arranged in parallel with each other.

10. The method according to claim 5, wherein the exposing of the photoresist in the step (c) is performed in an inclined manner.

11. The method according to claim 5, further comprising the steps of:
(f) fabricating a negative mold using the removed plating layer as an original form; and
(e) obtaining a molded product made of a polymer material using the negative mold.

12. An out-of-plane microneedle sheet comprising a plurality of microneedles according to claim 1 applied thereon.

13. The out-of-plane microneedle sheet according to claim 12, wherein the sheet comprises a through-recess formed therein so as to store or transport active agent or agents, the through-recess being adapted to fluidically communicate with the vertical groove of the microneedle.
